(19) United States
(12) Patent Application Publication
(10) Pub. No.: US 2002/0025514 A1
(43) Pub. Date: Feb. 28, 2002

(54) High throughput assay

(76) Inventors: Deborah D. Jaworski, Collegeville, PA (US); Monique F. Murray, King of Prussia, PA (US)

Correspondence Address: GLAXOSMITHKLINE, Corporate Intellectual Property - UW2220, P.O. Box 1539, King of Prussia, PA, 19406-0939, US

(21) Appl. No.: 09/896,304

(22) Filed: Jun. 29, 2001

Related U.S. Application Data
(60) Provisional application No. 60/215,817, filed on Jul. 5, 2000.

(51) Int. Cl. C12Q 1/70; G01N 33/53; G01N 33/567; G01N 33/569; C12N 5/06; C12N 5/16

(52) U.S. Cl. 435/5; 435/7.2; 435/7.22; 435/345

(57) ABSTRACT

A high-throughput live whole cell assay method involves measuring the effect of one or more test compounds on a characteristic, e.g., growth, of a cell culture in a multi-well vessel at one or more timepoints during the growth of these cells in culture, after the cells are contacted with the compound. This assay format permits the identification of test compounds that have an inhibitory or stimulatory effect, among others, on cell growth at different times during the growth phase of the cells. It has the advantages of using small volumes which make the assay suitable for automation and enables detection of test compounds with the desired effect that are often missed by conventional assays which assess test compound activity at a single timepoint.

### FIELD OF THE INVENTION

The present invention relates to the field of assays for the detection of novel compounds that have anti-microbial, particularly anti-bacterial, activity.

### BACKGROUND OF THE INVENTION

In the pharmaceutical industry, high-throughput screening (HTS) assays are critical for drug discovery or new target discovery. Screening assays, in general, involve the process of discriminating from among a host of test compounds, those that have a desired activity against a target vs. those that are inactive. HTS assays are built around cells or purified molecular targets, such as receptors, enzymes, etc. Some cell types used in HTS assays are pathogenic bacteria, transformed mammalian cells and genetically engineered bacterial. Problems have been noted in current HTS assays [see, e.g., S. J. Fox et al, High Tech Bus. Decision, March 2000, pp. 40-44] include poor quality and inconsistency in reagents and plates, technological issues relating to liquid handling, assay formats, and the expense of the assays. Current manual assays, such as the MIC assay [see, e.g., MICROBIOLOGY, 2nd edit., L. Prescott et al, eds., WCB Publ., Dubuque, Iowa 1993] measure the ability of a test compound to inhibit growth of a microorganism by a single timepoint, usually about 3 hours or between 18-20 hours. The usual volumes required for testing in a MIC assay are about 1 ml to about 50 mls or greater. Additional disadvantages for such “low-throughput” manual assays are that the microorganism can grow too rapidly for the detection of less potent antimicrobial test compounds. Current HTS assays require expensive reagents and the need to prepare a recombinant microorganism that expresses a particular enzyme. One significant problem with current HTS assays is the difficulty in obtaining consistent data for small volumes of cultures, suitable for use in automated assays.

There remains a need in the art for additional assay methods which permit more sensitive identification of larger numbers of active compounds against desired targets, particularly agents of disease, e.g., pathogens, in a less time-consuming and less expensive manner.

### SUMMARY OF THE INVENTION

In one aspect, the present invention provides a high-throughput assay comprising the step of measuring the effect of a test compound on a biological or biochemical characteristic of a selected microorganism culture in a multi-well, microtiter vessel at one or more timepoints during the growth of the culture, after the culture is contacted with the test compound. In another embodiment, the invention provides a high-throughput live whole cell assay method that comprises the step of measuring the effect of a test compound on the growth or kinetics of growth of a whole cell culture in a multi-well vessel at multiple timepoints during the growth of the cells in culture, while the cells are in contact with the compound. Test compounds having an effect, e.g., an inhibitory, cidal or stimulatory effect, on the selected characteristic, such as cell growth, at one or more different times during the cell growth cycle are detected.

In another aspect, the HTS assay described above preferably measures the desired effect at four or more timepoints and at even or uneven intervals. More preferably the timepoints are 0,4 hours, 8 hours, 12 hours, 16 hours, 20 hours and 24 hours. The information provided by the HTS assay permits the identification of compounds, which have the desired effect in smaller volumes of cultures than are ordinarily used in standard screening assays. Additionally, this embodiment of the assay permits the identification of compounds which have the desired effect at different points in the growth stage of a particular cell, and which may be missed by conventional single-timepoint assays. In an additional embodiment, the assay may be automated.

In another aspect, the invention provides a compound having a desired effect, e.g., inhibitory, cidal, stimulatory, etc. on a biological or biochemical characteristic, e.g., the growth of a selected cell type identified by the above method.

In a further aspect, pharmaceutical compositions containing such compounds, preferably compounds that inhibit pathogenic microbial (e.g., bacterial or fungal) growth, are provided herein.

In another aspect, compositions for enhancement of microbial growth, such as for enhancing the fermentation of microorganisms that produce antibiotics which are identified by the method above, are provided.

Other aspects and advantages of the present invention are described further in the following detailed description of the preferred embodiments thereof.

### BRIEF DESCRIPTION OF THE DRAWINGS

FIG. 1 is a scatterplot of optical density measurements made at an 8 hour timepoint using an assay of this invention. The boxed section of the figure illustrates the number of test compounds active in the above assay, which compounds were not detected in a conventional one timepoint MIC assay.

FIG. 2 is a scatterplot of optical density measurements made at a 20 hour timepoint from an assay of this invention. The small box also shows test compounds active in inhibiting growth of the microorganism, which were not identified in an MIC assay.

FIG. 3 is a graph showing data for the multi-timepoint assay of this invention, using compounds that are positive in a short three hour assay (Lux) and in a 20 hour conventional MIC assay (% inhibition plotted against the timepoints of 8 hours, 12 hours, 16 hours, 20 hours and 24 hours). Test compounds are identified by the indicated symbols in the graph.

FIG. 4 is a graph showing data (% inhibition plotted against the timepoints of 8 hours, 12 hours, 16 hours, 20 hours and 24 hours) for the multi-timepoint assay of this invention, using compounds that demonstrated greater than 95% inhibition at 10 μM, and demonstrated less than desirable results in the MIC assay, i.e., inhibition at greater than 50 μM. Test compounds are identified by the indicated symbols in the graph.

### DETAILED DESCRIPTION OF THE INVENTION

The present invention provides an improvement in high throughput assays that permits the identification of useful test compounds, which have an effect on (e.g., which inhibit or stimulate) a characteristic of a selected microorganism culture, preferably a culture in a volume small enough for automated assay in microtiter vessels. In certain embodiments, this assay permits the identification of compounds having the desired effect at selected times during the growth of the target culture. This assay can thereby discriminate among compounds, which are active at different (e.g., the early or late) stages of culture growth or are active at different culture densities. Thus, in some embodiments, this assay allows identification of useful test compounds that may be routinely missed by conventional single-timepoint assays. The present invention permits the automation and roboticization of the high throughput assay, such that components of the assay may be more accurately dispensed consistently at volumes not possible in manually performed assays. Further, this assay utilizes inexpensive reagents and methods for accomplishing the screening, and produces considerably more information and identification of compounds than are possible in other prior art assays.

The high-throughput assay method of the present invention comprises the step of measuring the effect of a test compound on some detectable characteristic of a cell culture in a small volume of culture in a multi-well, microtiter vessel.

As used herein, the term “microorganism culture” or “cell culture” is used generically to represent eukaryotes, prokaryotes and protozoans, particularly pathogenic strains of such cells or microorganisms. These cultures may be live whole cell cultures, cultures of spheroplasts, cultures of protoplasts, cultures of spores, or mixtures of at least one of these cell types with a non-cellular microorganism, e.g., a virus. For example, the cell culture may include bacterial cells, particularly pathogenic bacterial cells. For example, the cell culture may include fungal cells, particularly pathogenic yeast cells. In certain embodiments, the cell culture may contain plant cells, insect cells, or mammalian cells, such as cancer cells. Alternatively, the cells may be genetically engineered pathogenic bacterial or mammalian cells, which are engineered to contain or express heterologous DNA sequences or proteins. Mixtures of all such cells and microorganisms are contemplated in a culture, as are mixtures of one or more of these microorganisms infected with, or admixed with, one or more viruses. For instance, eukaryotic cells with intracellular pathogens, e.g., Listeria, etc., are contemplated for use as the microorganism cultures to be assayed by this HTS. The term “cell” or “cell culture also encompasses mammalian lines, e.g., immortalized cancer cells, and includes mammalian cells infected with pathogens, such as viruses.

Preferably, the term relates to pathogenic cellular microorganisms such as bacteria, yeast and fungi, which cause disease, particularly in mammals. Among the suitable cells for use in this assay are members of any of the genera Streptococcus, Staphylococcus, Bordetella, Corynebacterium, Mycobacterium, Neisseria, Haemophilus, Actinomycetes, Streptomycetes, Nocardia, Enterobacter, Yersinia, Francisella, Pasteurella, Moraxella, Actinobacter, Erysipelothrix, Branhamella, Actinobacillus, Streptobacillus, Listeria, Calymmatobacterium, Brucella, Bacillus, Clostridium, Treponema, Escherichia, Salmonella, Klebsiella, Vibrio, Proteus, Erwinia, Borrelia, Leptospira, Spirillum, Campylobacter, Shigella, Legionella, Pseudomonas, Aeromonas, Rickettsia, Chlamydia, Borrelia and Mycoplasma, and further including, but not limited to, a member of the species or group, Group A Streptococcus, Group B Streptococcus, Group C Streptococcus, Group D Streptococcus, Group G Streptococcus, Streptococcus pneumoniae, Streptococcus pyogenes, Streptococcus agalactiae, Streptococcus faecalis, Streptococcus faecium, Streptococcus durans, Neisseria gonorrheae, Neisseria meningitidis, Staphylococcus aureus, Staphylococcus epidermidis, Corynebacterium diptheriae, Gardnerella vaginalis, Mycobacterium tuberculosis, Mycobacterium bovis, Mycobacterium ulcerans, Mycobacterium leprae, Actinomycetes israelii, Listeria monocytogenes, Bordetella pertusis, Bordatella parapertusis, Bordetella bronchiseptica, Escherichia coli, Shigella dysenteriae, Haemophilus influenzae, Haemophilus aegyptius, Haemophilus parainfluenzae, Haemophilus ducreyi, Bordetella, Salmonella typhi, Citrobacter freundii, Proteus mirabilis, Proteus vulgaris, Yersinia pestis, Klebsiella pneumoniae, Serratia marcessens, Serratia liquefaciens, Vibrio cholera, Shigella dysenterii, Shigella flexneri, Pseudomonas aeruginosa, Francisella tularensis, Brucella abortis, Bacillus anthracis, Bacillus cereus, Clostridium perfringens, Clostridium tetani, Clostridium botulinum, Treponema pallidum, Rickettsia rickettsii and Chlamydia trachomitis

By the term “test compound” is meant any synthetic or naturally-occurring chemical compound and any recombinantly-produced molecule, including compounds contained or produced in combinatorial libraries, or compounds for which the structures were designed by computer or three dimensional analysis. Generally, pharmaceutical companies have batteries of unknown or undeveloped compounds, which may be “test compounds” for purposes of this invention. Such test compounds may be used in the method of this invention alone, in an inert buffer, e.g., saline, or may contain a solvent. Suitable solvents include dimethylsulfoxide (DMSO), alcohols, such as methanol and ethanol, or aqueous solutions, such as water, culture medium, etc. Other useful solvents may be selected from among those known in the art [See, e.g., other solvents discussed in ORGANIC CHEMISTRY, 3rd edit., R. Morrison et al, eds., Allyn and Bacon, Inc., Boston, Mass. 1973 and MEDICINAL CHEMISTRY, 2nd edit., T. Nogrady edit., Oxford University Press, New York 1988].

The term, “selected characteristic” or “biological or biochemical characteristic”, of the test compound on the cell culture is intended to define a wide variety of physical, biological or biochemical characteristics of the above-defined cell types. For example, one such characteristic is growth, including, for instance, enhancement of growth, inhibition of growth, kinetics of growth or stasis. Growth characteristics best measured in the assay over multiple timepoints. Other characteristics include, without limitation, isotropy, the electromagnetic spectrum of the cell, the morphology of the cells in culture, sporulation, lysis of the cells, tumbling, polarization, and refractive index. Such a characteristic may also include integrity of the cell wall or nucleus, efficiency of the cell's production of fermentation products or efficiency of recombinant expression of heterologous proteins, etc.

By the term “the effect” or “desired effect” is meant the result of contact of the cell with the test compound on the above-identified characteristic(s) of the cell. The effect can be detected by spectrophotometric means, by optical density or an effect that is detectable because it causes an alteration in the electromagnetic spectrum. Thus, as one embodiment, the effect to be measured is an inhibitory effect or cidal effect (static) on growth of the cells that can readily be detected by optical density measurements. As the test compound inhibits the growth of the cells in the culture, the density of the culture decreases. Density of the culture is most readily determined by OD measurements. Additionally, other effects measurable by OD include a growth stimulatory effect by the test compound, wherein as the growth of the cells exposed to the test compound increases, the OD increases. Based on the number of timepoints measured by the assay, these effects may be detected at one or more stages of cell growth. The assay may also permit the determination of other effects, which are detectable by spectrophotometric assays, other than optical density. For example, any change in the culture that is detectable optically, such as a calorimetric change or fluorescence, or is due to a change in the isotropy or in the electromagnetic spectrum may also be an effect measurable and detectable by this assay. Such other effects of the test compounds include, without limitation, differences in morphology of the culture, sporulation, lysis, tumbling, polarization, or refractive index, among other characteristics in the cell cultures contacted with various test compounds.

According to this invention, a test compound is contacted with a microorganism culture in a multi-well vessel. The vessel may contain any desired number of wells. A typical multi-well, microtiter vessel useful in this assay is a multi-well plate including, without limitation, 10-well plates, 96-well plates, 384-well plates, 1536-well plates, and plates having greater than 1536 wells. Opaque plates may also be used, which can contain volumes of about 10 microliters. Alternatively, an array of tubes may be used, depending on the volume desired. The only variable in the method depending on the number of wells or tubes is the volume, which may be contained by each well.

The total volume for each well useful in the HTS assay of the invention comprises generally the cell culture (the cells or other particles and culture medium) and the amount of test compound (including any amount of solvent contained therein) added. Significantly for the automation of this assay, the total volume is less than that required by a conventional MIC assay, i.e., less than about 1 milliliter. More preferably, the volume contained in each well of the plate is a total volume of microbial culture and test compound of less than about 0.5 mil. The total volume of microbial culture and test compound may be less than about 500 microliters. More preferably, each well of said vessel contains a total volume of microbial culture and test compound of less than about 200 microliters. Still more preferably each well of said vessel contains a total volume of microbial culture and test compound of less than about 100 microliters. In yet another embodiment, the total volume in each well is less than about 50 microliters. Desirably, the volume contained in each well is less than about 10 microliters. Even more desirably, each well of said vessel contains a total volume of microbial culture and test compound of less than about 1 microliter. It is an unexpected advantage of this invention that such small volumes containing the abovedefined microorganism cultures, including particularly, live whole cell cultures, can be utilized in this assay.

According to the method of this invention, the number of cells or other particles in each well of the multi-well plate is desirably at least 10³ cells/μL culture or at least about 10³ cfu/μL culture. In another embodiment, the cell culture contains a concentration of about 5×10⁴ cfu/μL culture or 5×10⁴ cells/μL culture. In still another embodiment, the cell number added to each well is 2×10⁵ cells/μL culture. In yet other embodiments of this method, the cell culture contains a concentration of about 10⁷ cfu/μL culture or 10⁷ cells/μL culture. Other cell numbers may be contained in the wells, depending upon the sensitivity of the test compounds, the size of the wells, the characteristic to be measured, and the detection methods employed in the assay. The cells or other microorganism particles in the culture are generally in a growth medium suitable to the selected cells. There are any number of commercially available growth media, which may be selected for the cell type. In the examples below, the selected growth media for the selected cells S. aureus is brain heart infusion (Difco). It should be understood that selection of a suitable medium is well within the skill of the art. See, e.g., commercial media catalogs such as those produced by Difco and BBL, as well as the media described in texts, such as Bergey's Manual of DETERMINATIVE BACTERIOLOGY, 8th edit., Buchanan et al, eds, The Williams & Wilkins Co., Baltimore, Md. 1975; MICROBIOLOGY, INCLUDING IMMUNOLOGY AND MOLECULAR GENETICS, 3rd edit, Davis et al, eds., Harper & Row Publ., Philadelphia 1980; and MEDICAL MYCOLOGY, J. W. Rippon, Ph. D., edit, W. B. Saunders Co, Philadelphia 1974, among others.

The total volume of cell culture and test compound is generally present in a standard dilution or ratio. Generally, the ratio of the compound to cell culture is limited only by the identity and amount of the solvent, if any, in which the test compound is present and upon the concentration of the test compound that is desired. Generally, the test compound is used at a concentration ranging from about 0.1 μM to about 10 μM, although higher and lower concentrations may be employed. The 10 μM concentration of the test compound is at least 10 times greater than concentrations useful in manual assays. However, other concentrations may be used depending upon the potency of the compound's effect on the selected cells used in the assay. If the compound is in any solvent (e.g., DMSO) other than the culture broth, the concentration of the solvent in the final reaction mixture is preferably limited to about 5% of the total volume. More preferably, the solvent concentration, if any, in the test compound is less than about 2% of the total volume of the well. Suitable solvents for test compounds may be readily selected by the art from standard chemistry texts. See, e.g., MEDICINAL CHEMISTRY, 2nd edit., T. Nogrady edit., Oxford University Press, New York 1988.

As stated above, the ratio of test compound to cell culture can be any standard dilution, such as 1:50. For example, it is possible to dilute the compound between ratios of about 1:500 to about 1:5, as long as the compound is in the appropriate solvent (e.g., in the case of 1:5 dilution, the compound must be in growth medium), and is appropriately concentrated. Still other ratios can be used. In a preferred embodiment, the total volume of cell culture and test compound added to each well in a standard 1:50 dilution is generally about 1 μL of 10 μM compound or less for every 49 μL of cell culture in the wells.

In the practice of the HTS assay, preferably the cell culture is established from frozen cells, placed in the medium and grown at least for about 8 to 12 hours (i.e., “overnight”) at a suitable temperature for normal growth of that cell type. Such normal growth temperatures may be readily selected based on the known growth requirements of the selected culture. For example, the growth/incubation temperature for S. aureus, which is illustrated in the examples below as a selected cell culture, was about 37° C. Preferably, during the establishment of the culture and particularly during course of the method, the cell culture is incubated in a controlled CO₂/N₂ humidity suitable for growth of the selected cells before and after contact with the test compound and measurement at the selected timepoint(s). The humidity of the incubation is controlled to minimize evaporation from the microtiter vessel, and permit the use of small volumes. Alternatively, or in addition to controlling humidity, the vessels may be covered with lids in order to minimize evaporation. Selection of the incubation temperature depends upon the identity of the cells, primarily. In the example below, for instance, the CO₂/N₂ humidity was about 30% for a 50 μL final volume with S. aureas as the cell culture in an incubation temperature of about 37° C. Selection of the percent humidity to control evaporation is based upon the selected volume of the vessel and concentration and volume of the test compound and cell culture in the vessel, as well as upon the incubation temperature. Thus, the humidity may vary from about 10% to about 80%. It should be understood that selection of a suitable incubation temperature, and time of incubation prior to initiation of the assay method and selection of controlled humidity is well within the skill of the art. See the texts cited immediately above. The cells, once incubated, are preferably diluted to a suitable cell number, as described above, in the same medium.

The test compound may be added to the cell culture immediately after the culture is established in an initial incubation, or the test compound may be added to the cell culture at a selected time during the course of the assay. Further, according to this assay, the microtiter plates with both test compound and cell culture are incubated at a desired temperature, which in one embodiment of this method, is the same initial incubation temperature for the cell culture. Alternatively, the incubation temperature may be varied during the course of the assay method, as desired to observe temperature fluctuation effects upon the test compound and cell culture characteristics, collectively or individually. As the temperature fluctuates in this embodiment, the humidity may be adjusted to prevent evaporation.

In one variant of the assay of this invention, each well may contain a single cell culture contacted with a single test compound. In another variant, each well may contain multiple different test compounds in the desired dilution. Another alternative is that each well may contain more than a single type of cell culture and each well is contacted with a different test compound, or with multiple test compounds.

According to one embodiment of this invention, the plates containing test compound and culture are removed from incubation and the effect of the test compound is measured at one timepoint or at multiple timepoints during the growth of the culture, after the culture has been in contact with the compound. Alternatively, such measurement(s) can occur while the cell is in contact with the compound. In one embodiment, the assay involves removing the test compound from contact with the culture at a selected time after said culture has been in contact with said test compound and before measurement of the desired effect. Such removal may be effected by rinsing the cells gently or by some other gentle procedure. Still other embodiments involve repeatedly contacting the culture with one or more test compounds, and/or repeatedly removing the test compounds prior to measurement, or contacting the culture with increasing dosages of the test compounds. Still another modification of the HTS assay includes adding additional components to the culture so that more than a single effect may be detected by the assay. For example, polynucleotide or protein probes may be added to the culture along with the test compound to detect the effects of the test compound on various genes and proteins normally present during the growth of such cells.

Whatever variant of well contents is selected for measurement, it may be preferred to shake the wells prior to each measuring step. With regard to the measurement of the desired effect, more than a single measurement or type of measurement may be employed to detect the effect(s) of the test compound(s) on the culture. The measuring steps may be repeated for multiple different test compounds on the same culture, or repeated for the same test compounds on multiple different cultures. The assay may repeat the same or different measuring steps for multiple different test compounds on the same culture. Alternatively the assay may include repeating the same or different measuring steps for the same test compounds on multiple different cultures.

In one embodiment, a single timepoint measurement is taken using this assay. Single timepoints are more useful to measure the effect of the compound on certain characteristics, such as morphology. More preferably, where the characteristic to be observed, is a kinetic characteristic changeable over time, e.g., growth stimulation or inhibition, the total amount of time that the assay is performed is over a 24 hour growth period. The number of timepoints at which the test compound effect is measured is at least four, including an initial measurement at timepoint 0. The intervals between the measurement of the effect may be regular, evenly spaced intervals. Alternatively, the intervals may be unevenly spaced intervals. Generally, the doubling time of the cells is useful in determining the time intervals. The doubling time may be controlled by nutrient deprivation, i.e., by adjustment of the culture medium to either increase or decrease culture doubling time. It is desirable that the intervals between measurements span from between 2 to 8 hours. In one preferred embodiment, the time between measurements is at least 3 hours. In another preferred embodiment, the time between measurements is at least 4 hours.

In a particularly preferred embodiment of the method, the timepoints for measurement occur at 0 hour, 4 hours, 8 hours, 12 hours, 16 hours, 20 hours and 24 hours. Still other embodiments may include 0 hour, 5 hour, 8 hours, 10 hours, 12 hours, 14 hours, 16 hours, 18 hours, 20 hours and 24 hours. In a particular embodiment in which the number of cells in the starting culture is the minimal number (e.g., 10³ cells/L), the timepoint reading after the initial reading occurs at 6 hours. One of skill in the art may readily select the number of timepoints as well as the timepoint intervals, depending upon the cells and test compounds used as well as the effect desired to be observed. Generally, each timepoint provides different information about how the test compound effects a characteristic such a growth. For example, some compounds produce an effect early in cell growth. Others effect the cells at a later point in development. Thus, the assay permits the identification of test compounds, which may exhibit the effect at one time or at different times during the cell growth. Knowledge of the timing of the effect of a test compound is useful in the design of therapeutic, diagnostic, or prophylactic compositions containing these compounds, as e.g., antimicrobials. Similarly, such test compounds are useful in compositions and methods to enhance microbial growth and thereby enhance the production of microbially-produced, or fermented products, such as antibiotics, antifungals, etc. These compounds and the knowledge of their timing and mode of activity are also useful in research methods and compositions.

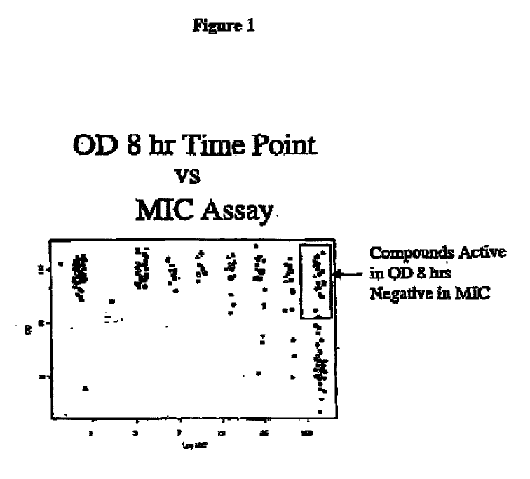

In the performance of the assay the measuring step is designed to identify and/or quantify the desired effect on the selected cell characteristic, e.g., inhibition or stimulation of the cell growth. For example, one desirable “measuring” step is the assessment of the cell density of the culture at each timepoint. In one embodiment, this can conventionally be performed on an optical density reader, using a suitable filter. Suitable filters for measuring optical density of cell cultures is generally about OD absorbency of 600. Typical OD values range from 400 to about 650 for various cell types. One of skill in the art may readily select the OD value, and such selection is not considered a limitation of this invention. Generally, for accurate OD readings, the cell density must be about 10⁷ cfu/μL in each well. Because cell growth increases the turbidity of the culture in the well, OD measurements increase with growth. OD measurements correlatively decrease with inhibition of growth or with cell death. The use of OD as the measuring step is desirable particularly for automation of the assay, using suitable robotics systems with plate scheduling software and peripherals to perform the movement of the plates from the incubator to the reader and vice versa, as well as any additional shaking of the plates prior to read. The subsequent data and analysis of the data (which involves subtracting the initial timepoint 0 background read from each other timepoint data) by plotting a growth curve based on OD measurements produces an analysis similar to those shown in FIGS. 1-4 herein.

Although OD absorbance is a presently convenient detection or measure method for use in the HTS assay of this invention where the effect is upon the density of cells in the culture, alternatively this assay may use another measuring step to detect effects on growth; alternatively, another measuring step may be more suitable to detection of characteristics other than growth. Preferably another spectrophotometric measurement process is used for measuring a detectable signal or reporter, indicative of cell growth or inhibition. For example, such a measurement step may involve the determination of fluorescence intensity, fluorescent resonance energy transfer, time resolved fluorescence, capillary electrophoresis, homogeneous time-resolved fluorescence, fluorescence activated cell sorting, fluorescence correlation spectroscopy, ion channel probes and phosphor imaging. To utilize such measurement steps, the cells are generally labeled prior to the assay with a detectable tag, which provides the detectable signal, either directly or indirectly. For instance, the tag may be a fluorescent label, or a luminescent label. Such labels may be a lux gene, a luciferase enzyme, a gluconase enzyme, a galactosidase enzyme, a radiolabel or any other detectable gene product. Other labels may require the addition of another compound to generate the detectable signal. Alternatively, the cells may be engineered to express a “signal protein”, e.g., GFP, and the absence or decrease in the expression and detection of such protein would be indicative of the inhibition or stimulation of cell growth or rapid cell death. Any number of additional, and conventionally employed, label systems may be adapted to the method of this invention. One of skill understands that selection and/or implementation of a label system involves only routine experimentation.

Other measurement steps may be used to detect changes in the cell culture unrelated to growth and density, e.g., polarization, tumbling, refractive index, etc., as discussed above. Conventional measurement apparatus and protocols for detection any changes in the electromagnetic spectrum or optical characteristics of cells are known in the art and may be readily employed to detect these other effects.

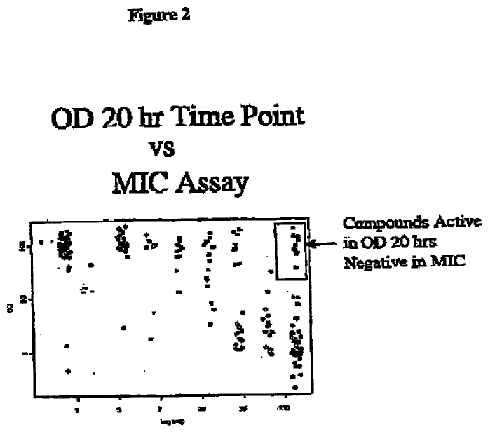

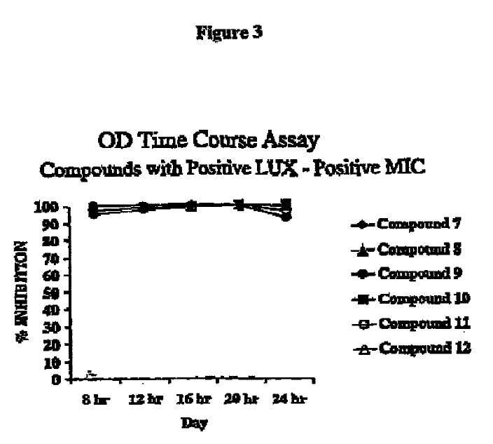

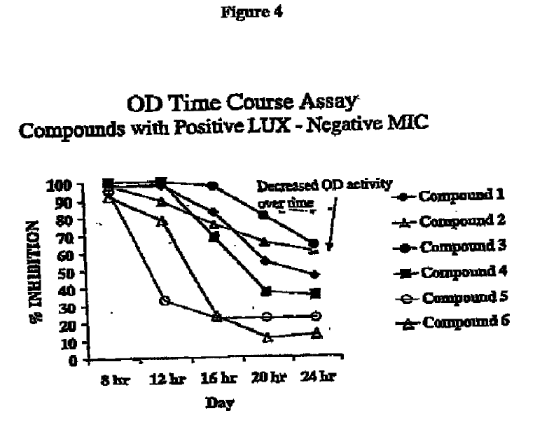

As described herein and with reference to FIGS. 1-4, an advantage of this high throughput assay is that it permits the identification of more useful test compounds than do other prior art assays. FIGS. 1 and 2 demonstrate the detection of test compounds “missed” by conventional one timepoint assays, but identified by this assay. Further, FIGS. 3 and 4 indicate that the data generated by the present assay provides additional information regarding when in the growth phases of the particular cell a particular test compound is maximally useful. Further, this assay permits the rapid screening of a larger number of test compounds (i.e., one test compound per well in a multi-well plate) than do current prior art assays.

Thus, as still another aspect of this invention, compounds that demonstrate the desired effect on a selected cell characteristic, e.g., inhibition or stimulation of cell growth or growth kinetics in culture, can be identified for use in various compositions. For example, compounds noted to inhibit microbial growth may be used in pharmaceutical compositions for the treatment of disease. Such pharmaceutical compositions will generally contain an effective amount (i.e., a disease-inhibiting or reducing amount) of one of the test compounds in a physiologically acceptable carrier, such as saline or other carriers known in the art. As used herein, the term “disease(s)” generally means microbial infections, particularly bacterial or fungal infections, and ailments related to such infections. Illustrative examples include, without limitation, staphylococcal infections including, but not limited to, infections of the upper respiratory tract (e.g., otitis media, bacterial tracheitis, acute epiglottitis, thyroiditis), the lower respiratory tract (e.g., empyema, lung abscess), the cardiac system (e.g., infective endocarditis), the gastrointestinal tract (e.g., secretory diarrhea, splenic abscess, retroperitoneal abscess), the Central Nervous System (e.g., cerebral abscess), eye (e.g., blepharitis, conjunctivitis, keratitis, endophthalmitis, preseptal and orbital cellulitis, dacryocystitis), the kidney or urinary tract (e.g., epididymitis, intrarenal and perinephric abscess, toxic shock syndrome), the skin (e.g., impetigo, folliculitis, cutaneous abscesses, cellulitis, wound infection, bacterial myositis), and the bones and joints (e.g., septic arthritis, osteomyelitis). Similarly diseases resulting from Streptococcus pneumoniae infection, such as otitis media, conjunctivitis, pneumonia, bacteremia, sinusitis, pleural empyema and endocarditis, and most particularly meningitis, such as for example, infection of cerebrospinal fluid are also included by the use of the term “disease”. This term also encompasses an infection caused by or related to any of the specifically identified genera or species of bacteria identified above. However, as stated above, the term “disease” may also include conditions caused by the unwanted growth of mammalian cells, e.g., cancer cells or virally-infected cells or the unwanted growth of fungal or yeast cells, e.g., candidiasis, etc. Disease may also be interpreted to include diseases of plants.

This assay is also useful in detecting test compounds for other pharmaceutical uses, e.g., compounds that inhibit the growth of cancer cells, and even the growth of virally infected cells, and compounds that stimulate the growth of certain desired microorganisms or cells (e.g., bacteria or fungi), such as those cells which produce desirable fermentation products. Test compounds that stimulate microbial growth are useful in growth enhancing compositions. These compositions may be used to enhance microbial fermentation processes, such as the production of antibiotics or antifungals, or in the microbial production processes involved in the manufacture of beer, wine, yogurt, and other foodstuffs and consumables, as well as in the microbial fermentations of useful chemicals, e.g., acetic acid, ethanol and the like. These compounds may be formulated into compositions suitable for each of these applications and employed in methods to enhance such processes by persons of skill in the art.

The assay of this invention may also be employed in the agricultural field, such as in the detection and identification of test compounds, which are effective to inhibit or stimulate the growth of certain plant cells or insect cells. The use of the test compound clearly depends on its effect on the selected culture used in the assay, and it is anticipated that given this disclosure, any number of effects on a selected cell culture may generate compounds of use in many fields of endeavor.

Still other compounds that have an effect on a cell characteristic other than growth are useful where the effect is useful for pharmaceutical, diagnostic, agricultural, research, industrial or other uses.

The following examples illustrate several embodiments of this invention. These examples are illustrative only, and do not limit the scope of the present invention.

### EXAMPLE 1

Novel Antibacterial OD Assay Protocol

S. aureus RN4220 [pKF1] are maintained as a frozen bacterial suspension. About one microliter of the frozen bacterial suspension is used to inoculate 100 mL of room temperature Brain Heart Infusion (BHI; DIFCO#0037178). The inoculate is then vortexed, and the cap loosened to ensure that the culture remains aerobic as it incubates overnight at 37° C.

To a 384 well clear plate (Costar#3072), about 1 μL of test compound in 100% DMSO is added. The following controls are used:

Control #1: 1 μL 100% DMSO (represents total possible growth)

Control #2: 1 μL 25 g/mL Mupirocin in 100% DMSO (represents no growth). Muripirocin is a commercial antibiotic that is effective and targets the isoleucyl tRNA synthetase enzymes, a known mode of action.

Control #3: 1 μL 25 g/mL Lincomycin in 100% DMSO (standard used to represent static compounds). Lincomycin is a drug that has a static mode of action. It does not kill the microorganism but rather stops growth, so that removal of the compound from the growth medium should allow the regrowth of the microorganisms. The importance of this static drug as a control in this assay is that it allows the assessment of effect over time. If the drug effect wears off after a certain period of time, the OD value should decrease as more organisms start to grow. This control permits a correlation with, and the identification of, test compounds that work early, but not late in the growth cycle.

Using room temperature BHI, the overnight culture is serially diluted as follows: overnight culture 1:10>1st dilution 1:10 >2nd dilution 1:10 >working culture. About 49 μL of working culture is added to all wells, and the wells are incubated for 24 hours at 37° C. Turbidity (OD600) is read at 6 timepoints: Time =0, 8 hours, 12 hours, 16 hours, 20 hours, and 24 hours.

In one experiment according to this example, the final concentrations of the test compounds in the plate during the course of the entire reaction times were as follows. Compounds tested as mixtures of 11 compounds per well were at a concentration of 9.1 μM per compound with 2% DMSO in a 50 μL volume. Compounds tested as single compounds per well were present at a concentration of 10 μM with 2% DMSO in a 50 μL volume. The cell concentration of S. aureus cells was 5×10⁴ CFU/mL. The concentration of Mupirocin was 0.5 g/mL and the concentration of Lincomycin was 0.5 g/mL.

The results of assays performed according to the above procedures can be observed in FIGS. 1 through 4. FIG. 1 shows the numbers of test compounds active in the above assay based on the 8 hour reading, which compounds were not detected in a conventional one timepoint MIC assay.

A MIC assay is an assay that mixes a desired number of microorganisms (about 0.06 μg/ml to about 64 μg/ml) in volumes of greater than about 1 mL and incubates the plates for between 18 to 20 hours at 37° C. The MIC is the concentration of drug that inhibits the growth of the bacteria, as determined visually. If all wells are clear, the MIC is taken as less than 0.06 μg/ml. If all wells have growth, the MIC is greater than 64 μg/ml.

The results of FIG. 1 demonstrate the ability of the assay of this invention to capture test compounds, which are active at different stages in the growth of a particular microorganism. Similarly FIG. 2 shows the numbers of test compounds active in the above assay based on the 20 hour reading. The small box also shows test compounds active in inhibiting growth of the microorganism, which were not identified in a MIC assay.

Comparing FIG. 1 with FIG. 2, one can determine that the compounds identified as active at 8 hours vs. 20 hours include some of the same test compounds, but also appear to include compounds which are different, i.e., some test compounds are active at 8 hours which are not active at 20 hours, and vice versa. Thus, this assay permits the identification of significantly more compounds useful for inhibiting the growth of this microorganism than identified by conventional assay procedures.

The results generated by the multi-timepoint assay of the invention may be compared with other conventional assays to provide critical additional information. For example, FIG. 3 is a graph showing data for the multi-timepoint assay of this invention, using compounds that scored positive in a short three hour Lux assay and in a 20 hour conventional MIC assay.

Briefly described, a Lux assay mixes the same concentration of cells as used in the above-described MIC assay, which cells contain the lux gene cassette [Carol L. Reid et al, “Microbial Detection and Two-dimensional Preservative Mapping using Lux-Based Bioluminescence”, in New Techniques in Food and Beverage Microbiology, Soc. for Appl. Bacteriology, edit. (1993)] with test compounds in a microtiter plate. The plate is incubated for 3 hours, and then cooled to room temperature. Substrate for the lux enzyme is added (0.1% octanol; BRI), diluted in ethanol to make a 0.01% working stock in the growth medium BHI. The plates are read in a luminometer and the percent inhibition (e.g., cell killing) is measured. The lower the number, the greater the inhibitory effect of the compound, because there are less enzymes to react with the substrate.

In this example, all compounds tested in the Lux assay showed greater than 95% inhibition at concentrations of 10 μM. All compounds when tested in the MIC assay showed inhibition at concentrations less than 3.12 μM. When tested in the multi-timepoint assay of the present invention, one can see that all six compounds showed high inhibition from 8 hours through 24 hours. Thus the present assay provides more information on the inhibitory effects of test compounds.

The additional information generated by the assay format of this invention is even more critical when the results of conventional assays are contradictory. FIG. 4 is a graph showing data for the multi-timepoint assay of this invention, using compounds that demonstrated greater than 95% inhibition at 10 μM, and demonstrated less than desirable results in the MIC assay, i.e., inhibition at greater than 50 μM. In contrast, the results of the multi-timepoint assay demonstrate the differences in the activities of the test compounds over time, thus permitting one to observe that a compound such as Compound 5 or 6, while appearing to be very active in Lux and negative in MIC, are actually highly inhibitory only up to about 8 and 12 hours, respectively and decline in effect rapidly thereafter. Similarly, Compound 4, despite its contradictory indications in the Lux and MIC assays, can be shown to have high inhibitory activity up to about 16 hours.

### EXAMPLE 2

Novel Antibacterial High Throughput Method Measuring Activity vs. Time

The method of the invention was employed for the rapid identification of compounds that exhibit antimicrobial activity. This method used whole cell cultures of S. aureus in 384 well microtiter plates, with a volume of 50 μL (49 μL cells+1 μL test compound) per well. The method was employed to screen of a 16,000 compound collection, with each compound being examined individually at a concentration of 10 μM. The HTP screen was executed on a core robotic system with the compounds assayed individually over a 24 hour period. Readouts of optical density at 600 and 405 nm were obtained every 4 hours to monitor bacteria growth. Over 1000 of the compounds were identified as having antibiotic activity at some point during the 24 hour test period. These compounds were identified and subsequently prioritized based on their chemical structure for use as antibiotic leads and/or tool compounds to identify novel antibacterial targets.

Numerous modifications and variations of the present invention are included in the above-identified specification and are expected to be obvious to one of skill in the art. Such modifications and alterations to the compositions and processes of the present invention are believed to be encompassed in the scope of the claims appended hereto.

What is claimed is:

1. A high-throughput assay method comprising the step of:
measuring the effect of a test compound on a characteristic of a selected microorganism culture in a multi-well, microtiter vessel at one or more timepoints during the growth of said culture, after said culture is contacted with said test compound.

2. The method according to claim 1 wherein said microorganism is selected from the group consisting of eukaryotes, prokaryotes and protozoans.

3. The method according to claim 1 wherein said microorganism culture comprises components selected from the group consisting of a live whole cell, a spheroplast, a protoplast, a spore; and a mixture of said cells with a virus.

4. The method according to claim 3, wherein said cell is selected from a bacteria cell, a plant cell, a mammalian cell, a fungal cell, and a mixture of one or more of said cells and a virus.

5. The method according to claim 4, wherein said cell culture is selected from the group consisting of pathogenic bacterial cells, pathogenic yeast cells, mammalian cells, pathogenic protozoans, genetically engineered pathogenic or mammalian cells.

6. The method according to claim 1, wherein said characteristic is selected from the group consisting of cell growth, isotropy, the electromagnetic spectrum of the cell, the morphology of the cells in culture, sporulation, lysis of the cells, tumbling, polarization, refractive index, integrity of the cell wall or nucleus, efficiency of the cell's production of fermentation products and efficiency of recombinant expression of heterologous proteins by the cell.

7. The method according to claim 1 wherein said effect is selected from the group consisting of inhibition of microbial culture growth, stimulation or enhancement of microbial culture growth, and any change in the culture detectable by any optical, electromagnetic or spectrophotometric assay.

8. The method according to claim 1, further comprising the steps of removing the test compound from contact with the culture at a selected time after said culture has been in contact with said test compound.

9. The method according to claim 1, wherein each well of said vessel contains a total volume of microbial culture and test compound of less than about 1 ml.

10. The method according to claim 9, wherein each well of said vessel contains a total volume of microbial culture and test compound of less than about 0.5 ml.

11. The method according to claim 10, wherein each well of said vessel contains a total volume of microbial culture and test compound of less than about 500 microliters.

12. The method according to claim 11, wherein each well of said vessel contains a total volume of microbial culture and test compound of less than about 200 microliters.

13. The method according to claim 12, wherein each well of said vessel contains a total volume of microbial culture and test compound of less than about 100 microliters.

14. The method according to claim 13, wherein each well of said vessel contains a total volume of microbial culture and test compound of less than about 50 microliters.

15. The method according to claim 14, wherein each well of said vessel contains a total volume of microbial culture and test compound of less than about 10 microliters.

16. The method according to claim 15, wherein each well of said vessel contains a total volume of microbial culture and test compound of less than about 1 microliter.

17. The method according to claim 9, wherein said volume comprises about 49 μLs of cells in a medium suitable for growth of said cells.

18. The method according to claim 9, wherein said volume comprises a test compound at a concentration of less than or about 10 μM.

19. The method according to claim 18 wherein said volume comprises about 0.1 μL of a 10 μM test compound and about 49.9 μLs of cells in said medium.

20. The method according to claim 9, wherein said volume comprises at least 10³ cells/μL.

21. The method according to claim 1, wherein said cell culture contains a concentration of at least about 10³ cfu/μL culture or 10³ cells/μL culture.

22. The method according to claim 21, wherein said cell culture contains a concentration of about 5×10⁴ cfu/μL culture or 5×10⁴ cells/μL culture.

23. The method according to claim 22 wherein said cell culture contains a concentration of about 10⁷ cfu/μL culture or 10⁷ cells/μL culture.

24. The method according to claim 20, wherein said volume comprises about 1 μL or less of said test compound at a concentration of about 10 μM with about 49 μLs of said microbial culture comprising at least 10³ cells/μL.

25. The method according to claim 1, which is an automated method.

26. The method according to claim 1, further comprising the steps of:
incubating said microorganism culture under controlled humidity before and after each said measuring step at a temperature suitable for growth of said microbial culture.

27. The method according to claim 1, further comprising the step of shaking said culture before each measuring step.

28. The method according to claim 1, further comprising the step of:
repeating said measuring steps for multiple different test compounds on the same culture.

29. The method according to claim 1, further comprising the step of:
repeating said measuring steps for the same test compounds on multiple different cultures.

30. The method according to claim 1, wherein said multi-well vessel is selected from the group consisting of 10-well vessels, 96-well vessels, 384-well vessels, 1536-well vessels, and vessels having greater than 1536 wells.

31. The method according to claim 1, wherein each well in said vessel contains a single test compound.

32. The method according to claim 1, wherein each well in said vessel contains multiple test compounds.

33. The method according to claim 1, wherein each well in said vessel contains a single microbial culture.

34. The method according to claim 1, wherein each well in said vessel contains multiple microbial cultures.

35. The method according to claim 1, wherein said measuring step comprises measuring the cell density of said culture at each said timepoint, wherein test compounds having an effect on said cell growth are detected by an increase or decrease in culture density.

36. The method according to claim 1, wherein said measuring step comprises a method of detecting any change in an optical or electromagnetic characteristic of said culture.

37. The method according to claim 1, wherein said measuring step comprises measuring a detectable signal indicative of said effect.

38. The method according to claim 37, wherein said microorganism culture is labeled with a detectable tag, which provides said signal.

39. The method according to claim 38, wherein said tag is selected from a fluorescent label, and a luminescent label.

40. The method according to claim 39, wherein said label is selected from the group consisting of a lux gene, a luciferase enzyme, a gluconase enzyme, and a galactosidase enzyme.

41. The method according to claim 1, wherein said measuring step comprises a spectrophotometric measurement.

42. The method according to claim 41, wherein said measurement is selected from the group consisting of fluorescence intensity, fluorescent resonance energy transfer, time resolved fluorescence, capillary electrophoresis, homogeneous time-resolved fluorescence, fluorescence activated cell sorting, fluorescence correlation spectroscopy, ion channel probes and phosphor imaging.

43. The method according to claim 1, wherein said timepoints occur in intervals occur over a 24-hour period.

44. The method according to claim 43, wherein said intervals span from between 2 to 8 hour intervals.

45. The method according to claim 43, wherein said timepoints comprise at least four timepoints including an initial timepoint.

46. The method according to claim 43, wherein said timepoints are 0,4 hours, 8 hours, 12 hours, 16 hours, 20 hours and 24 hours.

47. A compound identified by the method of claim 1.

48. A pharmaceutical composition comprising a compound of claim 47 in a physiologically acceptable carrier.
